(19) United States
(12) Patent Application Publication
(10) Pub. No.: US 2005/0209599 A1
(43) Pub. Date: Sep. 22, 2005

(54) Osteotomy plate with locking washers

(75) Inventor: Mark Brunsvold, Marco Island, FL (US)

Correspondence Address: DICKSTEIN SHAPIRO MORIN & OSHINSKY LLP, 2101 L Street, NW, Washington, DC, 20037, US

(73) Assignee: Arthrex, Inc.

(21) Appl. No.: 11/023,556

(22) Filed: Dec. 29, 2004

Related U.S. Application Data
(60) Provisional application No. 60/536,706, filed on Jan. 16, 2004.

(51) Int. Cl. A61F 5/00

(52) U.S. Cl. 606/87

(57) ABSTRACT

Method and apparatus for performing osteotomies using an osteotomy plate having locking washers. A bone is resected to form an opening that is held open by a tooth projecting from a bone plate. The bone plate is secured to the outer surface of the bone with self-tapping bone plate screws. Screws are inserted through holes into the bone plate which contain washers that are rotatable in the holes. The outwardly-tapered head of the screw spreads the split sleeve washer as it advances and locks the washer. Slots in the split-sleeve washer can be engaged by a driver in order to adjust the bone plate so that lays properly against the resected bone.

This application claims the benefit of U.S. Provisional Application No. 60/536,706 filed Jan. 16, 2004.

### FIELD OF THE INVENTION

The present invention relates to osteotomies, and more specifically to methods and apparatus for performing osteotomies using an osteotomy plate having locking washers.

### BACKGROUND OF THE INVENTION

Osteotomies are procedures that surgically reorient the alignment of a deformed bone. The aim of an osteotomy is to realign the bearing surfaces of a joint to allow normal areas to articulate, moving abnormal areas away from the load-bearing axis. In the leg, a femoral or tibial osteotomy may be indicated. High tibial osteotomies (HTO), for example, are indicated by early, medial joint-space narrowing, by early arthritis in patients who have had previous medial meniscectomy, or following rupture of the anterior cruciate ligament (ACL) in patients with pre-existing varus deformity, for example.

There are two schools of thought regarding osteotomy methods: the closing wedge method, and the opening wedge method. In the closing wedge method, removal of a bone wedge creates an angled gap in the bone. Part of the bone is left as a hinge at the apex of the angle. The hinge allows the gap to narrow, and the bone material on either side of the closed gap joins together.

In the opening wedge method, a cut is made across the bone. Part of the bone is left as a hinge, as in the closing wedge method. In contrast to the closing wedge method, however, the hinge allows the cut gap to open. The open wedge is filled with graft material.

The two methods are performed on opposite sides of the bone to give equivalent results. For example, when a given deformity would be corrected by performing the opening-wedge procedure on the medial side of a bone, an equivalent closing-wedge correction would be performed laterally.

The closing wedge method is the current standard, although several disadvantages are associated with the technique. The most significant disadvantages of the closing wedge method are: (i) disruption of the tibial-femoral joint; (ii) possible damage to neurovascular structures; and, (iii) disruption of the medial cortex, resulting in instability and nonunion between the upper and lower bone because of possible soft tissue interference. It is also difficult to compute the correct amount of bone to remove, and, therefore, several extra cuts may be required.

The opening wedge technique avoids or limits many of the disadvantages associated with the closing wedge method. Additionally, the medial, open-wedge HTO has the following advantages over the closed, lateral-wedge HTO: (i) speed; (ii) simplicity; (iii) ability to quickly change angle at any time during the procedure; and (iv) no fibular osteotomy is required.

Apparatus and methods for performing opening wedge osteotomies are disclosed in U.S. Pat. Nos. 5,620,448 and 5,749,875 issued to Puddu and assigned to Arthrex, Inc., the disclosures of which are incorporated herein by reference, particularly as they relate to operative method and insertion tools. A similar system is disclosed in U.S. patent application Ser. No. 09/872,100, filed Jun. 1, 2001, the disclosure of which is incorporated herein by reference, particularly as it relates to grafts and graft materials developed for insertion into osteotomy openings. The present invention represents improvements on prior art systems.

### SUMMARY OF THE INVENTION

The present invention provides a system for performing opening wedge osteotomies in which a bone is resected to form an opening on one side governed by a bony hinge on the other side. The system includes a bone plate having a projection in the form of a tooth extending from the plate. The tooth on the bone plate is provided to maintain the bone opening. The edges of bone at the bone opening engage opposed edges of the bone plate tooth to keep the resected bone propped open. The bone plate extends outwardly on either side of the tooth for engaging the outer surface of the bone. Holes through the extended portions of the bone plate receive cancerous or cortical plate screws for securing the bone plate in place against the bone. The bone plate preferably is formed of a metal, such as titanium alloy.

The holes in the bone plate are provided with internally-threaded, split-sleeve washers that are pivotable or rotatable within the holes. The plate screws are installed through the threaded openings in the split-sleeve washers. The plate screws feature an outwardly-tapered head that spreads the split sleeve washers with screw advancement. The plate screw heads are threaded to complement the internal threads of the split sleeve washers. A bone screw portion of each screw is self-tapping and threaded appropriately to provide optimal purchase in bone.

An opening wedge osteotomy is performed by resecting bone laterally to form an opening governed by a hinge of cortical bone. The resected bone is wedged apart, and the bone plate is installed such that the bone plate tooth maintains the bone opening. The plate screws are installed to secure the bone plate in position, the rotatable washers allowing the screws to be oriented at an angle other than perpendicular to the bone plate while allowing a finished position with the screw head flush with an outer surface of the bone plate. During advancement, the outwardly tapered head of the plate screws urges the split-sleeve washers to spread radially and thereby exhibit a locking capability by way of frictional interference between the radially outer surface of the washer and an inner wall of the bone plate. Slots formed in the outward face of the split-sleeve washers can be engaged by a driver to turn the washer in the event that adjustments are required.

Other features and advantages of the present invention will become apparent from the following description of exemplary embodiments of the invention with reference to the accompanying drawings.

### BRIEF DESCRIPTION OF THE DRAWINGS

FIG. 1 illustrates a plan view of a tibial opening wedge osteotomy plate according to the present invention;

FIG. 2 illustrates an elevation view of the tibial opening wedge osteotomy plate of FIG. 1 taken along lines A-A;

FIG. 3 illustrates a split sleeve locking washer according to the present invention;

FIG. 4 is a perspective illustration of the bone plate of FIG. 1 with a mal-rotated washer according to the present invention;

FIG. 5 illustrates a plan view of the anterior/posterior sloped opening wedge osteotomy plate according to the present invention;

FIG. 6 illustrates an elevation view of the anterior/posterior sloped opening wedge osteotomy plate of FIG. 5 taken along lines B-B;

FIG. 7 illustrates a plan view of a femoral opening wedge osteotomy plate according to the present invention;

FIG. 8 illustrates an elevation view of the femoral opening wedge osteotomy plate of FIG. 7 taken along lines C-C;

FIG. 9 illustrates a cancerous plate screw according to the present invention;

FIG. 10 illustrates the cross-section of the cancerous screw of FIG. 9 taken along lines D-D;

FIG. 11 illustrates the cross-section of the notched tip of the cancerous screw of FIG. 9 taken along line E-E;

FIG. 12 illustrates a top view of the head of the cancellous plate screw of FIG. 9;

FIG. 13 illustrates a cortical plate screw according to the present invention;

FIG. 14 illustrates the cross-section of the cortical screw of FIG. 13 taken along lines F-F;

FIG. 15 illustrates the cross-section of the notched tip of the cortical plate screw of FIG. 13 taken along lines G-G;

FIG. 16 illustrates a top view of the head of the cortical plate screw of FIG. 13;

FIG. 17 is a schematic representation of the resecting and measuring step in an osteotomy procedure using a tibial opening wedge osteotomy plate, such as that of FIGS. 1 and 2;

FIG. 18 is a schematic representation of the locking and drilling steps in the osteotomy procedure of FIG. 17;

FIG. 19 is a schematic representation of the drilling and screw measuring steps in the osteotomy procedure of FIG. 17;

FIG. 20 is a schematic representation of a screw insertion step in the osteotomy procedure of FIG. 17;

FIG. 21 is a schematic representation of positioning and locking a bone screw and washer in the osteotomy procedure of FIG. 17;

FIG. 22 is a schematic representation of inserting the locking guide into the washer slots in order to rotate the washer in the osteotomy procedure of FIG. 17;

FIG. 23 is a schematic representation of loosening and tightening of a screw in the osteotomy procedure of FIG. 17; and

FIG. 24 is a schematic representation of a completed installation of a tibial bone plate according to the present invention.

### DETAILED DESCRIPTION OF THE PREFERRED EMBODIMENT

The present invention provides techniques and apparatus for open wedge osteotomies. For simplicity, the methods described herein are with reference to, but not limited to, one type of osteotomy, the tibial opening wedge osteotomy.

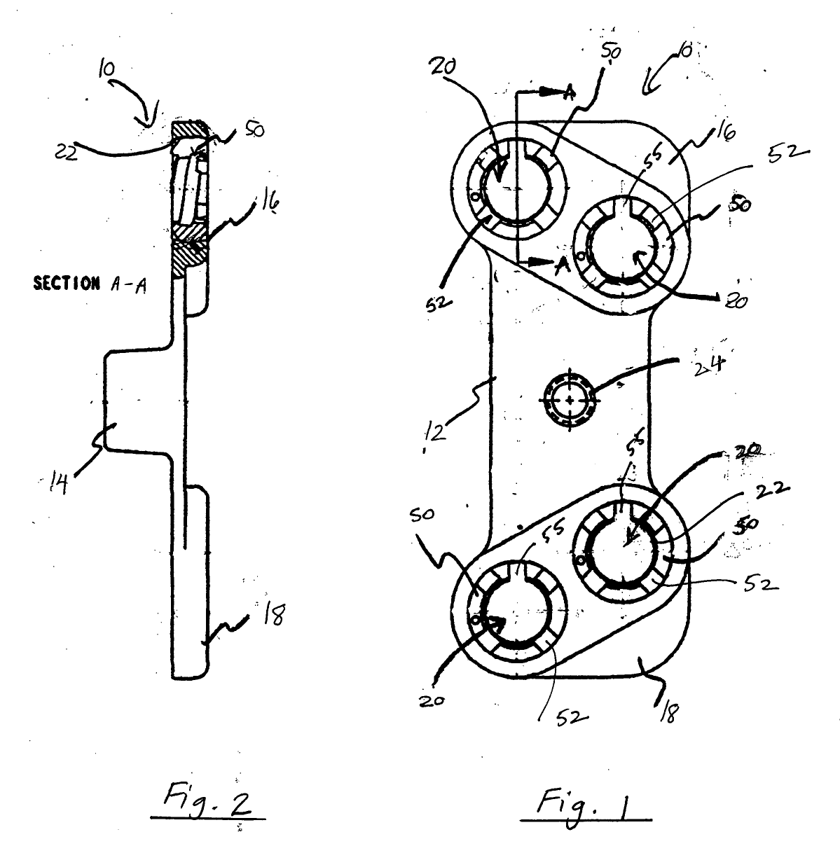

Referring now to FIGS. 1-2, a tibial opening wedge osteotomy bone plate 10 includes a plate 12 having a projecting tooth 14. Plate 12 has extensions 16, 18 on either side of tooth 14 with holes 20 formed in the extensions. Each of the holes 20 is defined by a radially-curved internal wall 22 formed in the plate extensions 16, 18. A threaded, split-sleeve washer 50 is fitted into each of the holes 20 in the plate 10. The washers 50 rotate freely in any direction within the hole 20, but are not removable from the hole 20 by finger pressure.

Bone plate 10 is formed of a metal, preferably titanium alloy. Preferably, the grain of the metal is oriented parallel with the longer dimension of the bone plate. A threaded central opening 24 is provided to receive an application bar (not shown) to facilitate handling of the bone plate 10.

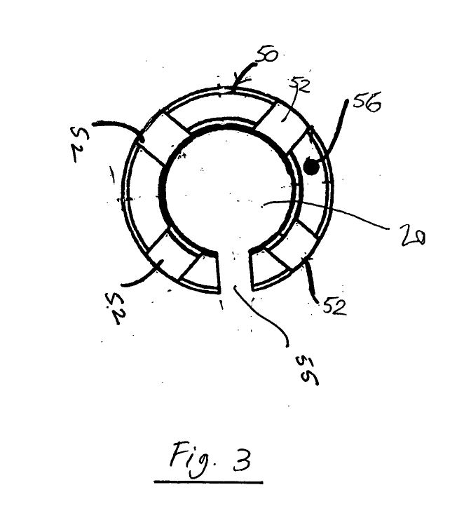

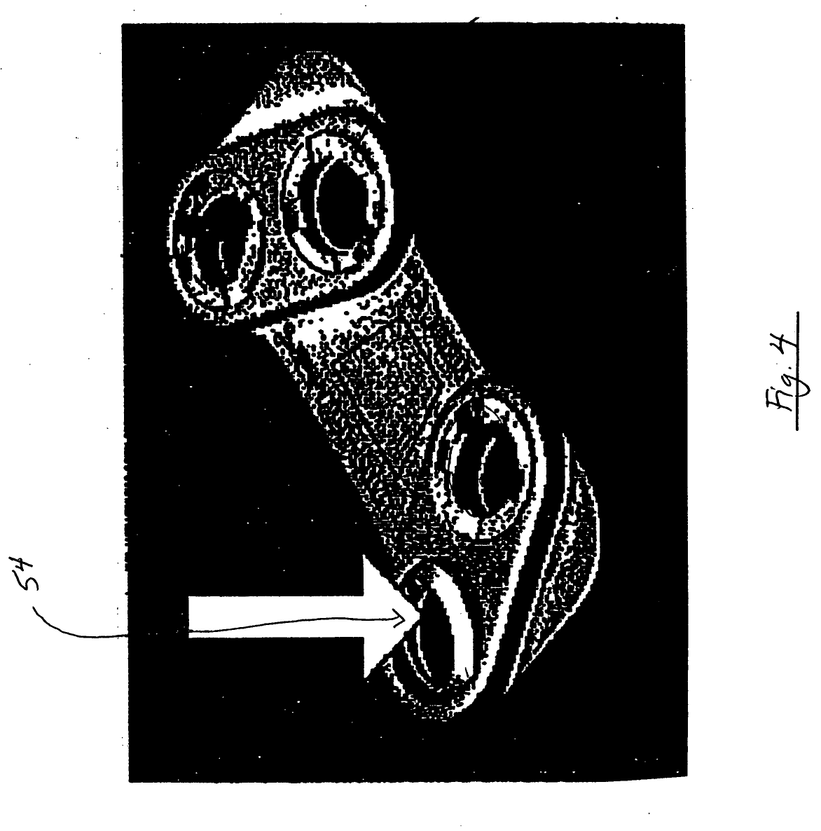

Referring to FIG. 3, a visualization mark, such as mark 56, is provided on each washer 50. Each washer 50 includes slots 52 formed in one side by toothed projections, and an opposing smooth side 54 (FIG. 4). FIG. 4 shows three of the washers 14 oriented with the slotted side facing up, and one washer mal-rotated so that the slotted side faces down, such that the smooth side 54 is visible in the drawing. The slots are configured to be engaged by a locking guide 138, described further below.

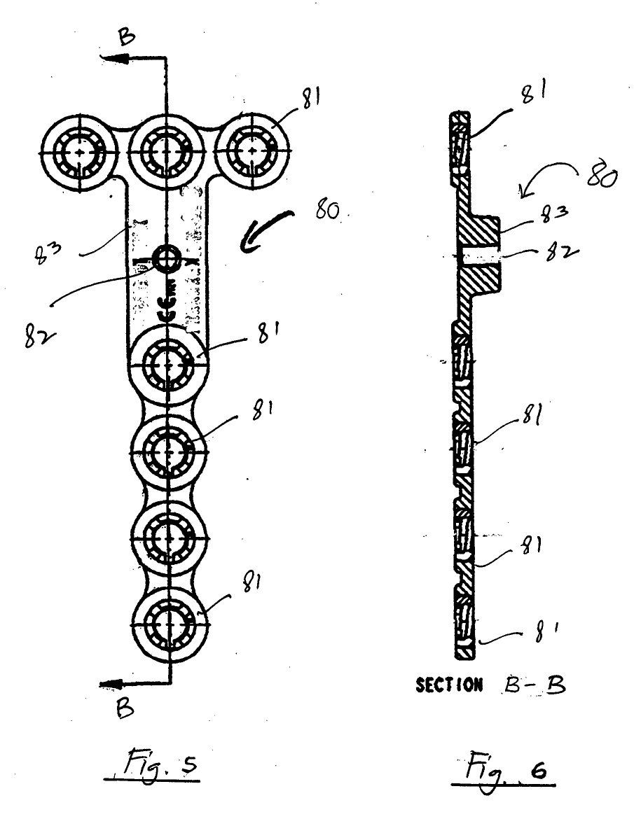

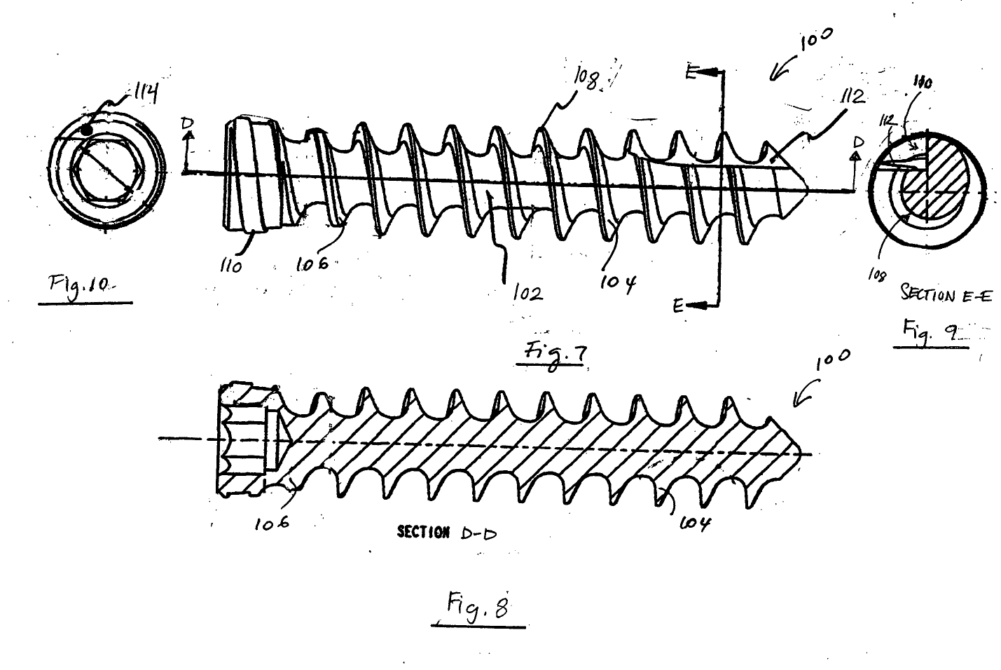

Bone plates of the present invention can be provided in various configurations depending on the indicated osteotomy to be performed. FIGS. 5-6 illustrate an exemplary embodiment of a tibial anterior/posterior (A/P) sloped osteotomy plate 70. FIGS. 6-7 illustrate an exemplary embodiment of a femoral opening wedge osteotomy plate 60. Each plate features the movable washers 50 described above in connection with FIGS. 1-3.

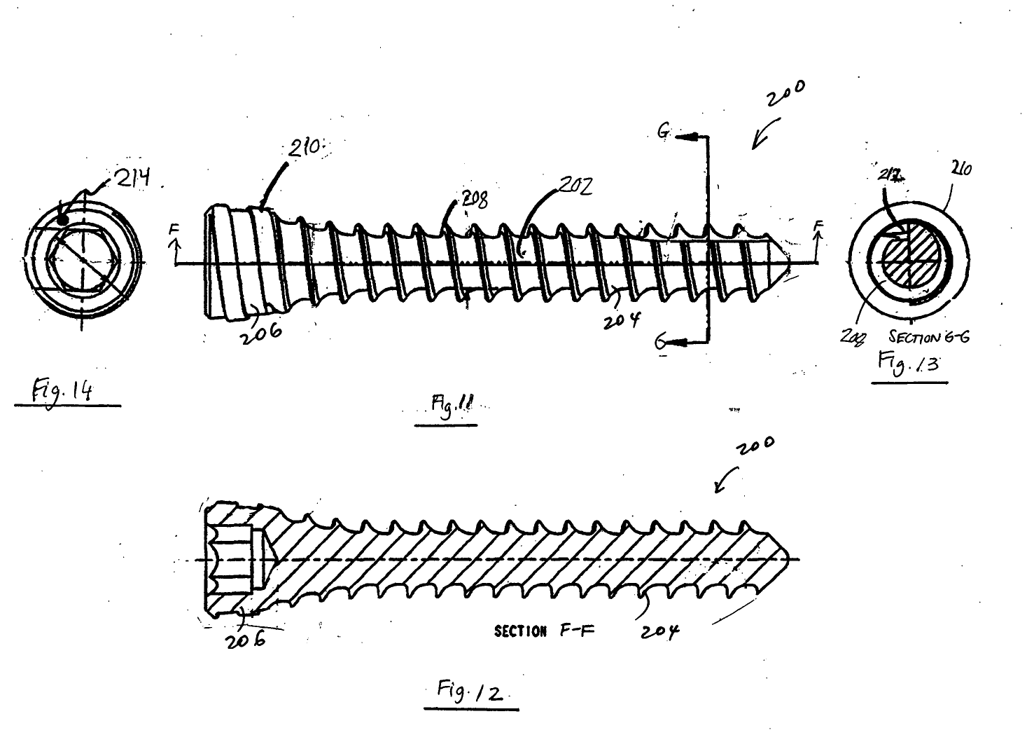

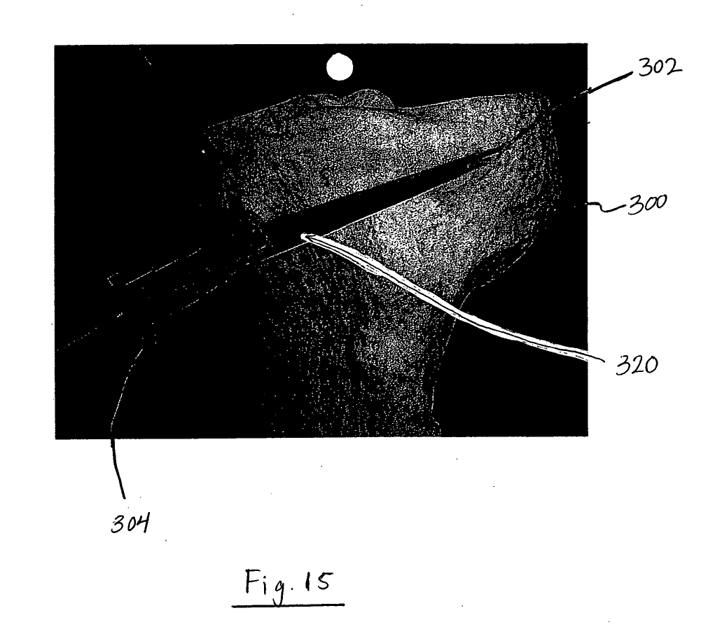

Referring to FIGS. 9-16, plate screws are used to secure a selected bone plate 10, 70, 80. FIGS. 9-12 illustrates a hex-drive cancellous plate screw 100. Plate screw 100 features a screw body 102 having a threaded bone portion 104 and a threaded screw head 106. The screw body 102 supports a cancellous bone thread 108 that extends over both the threaded bone portion 104, and a plate thread 110 that extends over the threaded screw head 106. The screw body 102 and cancellous bone thread 108 have substantially uniform outer diameters over the threaded bone portion 94. In the transition between the threaded bone portion 104 and threaded screw head 106, the outer diameter of plate thread 110 is reduced with respect to that of cancellous bone thread 108, and then tapers outwardly toward the head end of the plate screw 100. The reduced outer diameter of plate thread 110 facilitates initial engagement with the internal threads of washers 50 (FIGS. 1-3), followed by spreading of washers 50 by the outwardly tapering threaded screw head 106, as described further below. The threaded bone portion 104 features about nine thread flights 108, preferably at a thread pitch of about 0.108, suitable for use in cancellous bone. The threaded screw head 106 features about two thicker thread flights, preferably having a pitch of about 0.133, to complement the internal threads on washers 50. Referring to FIG. 11, preferably, a notch 112 is formed near the tip of screw 100 to facilitate self-tapping installation of screw 90 into bone. Referring to FIG. 12, a visual mark, such as black mark 114, is provided on the head of each screw 100. Bone screw 100 is made of metal, preferably titanium alloy.

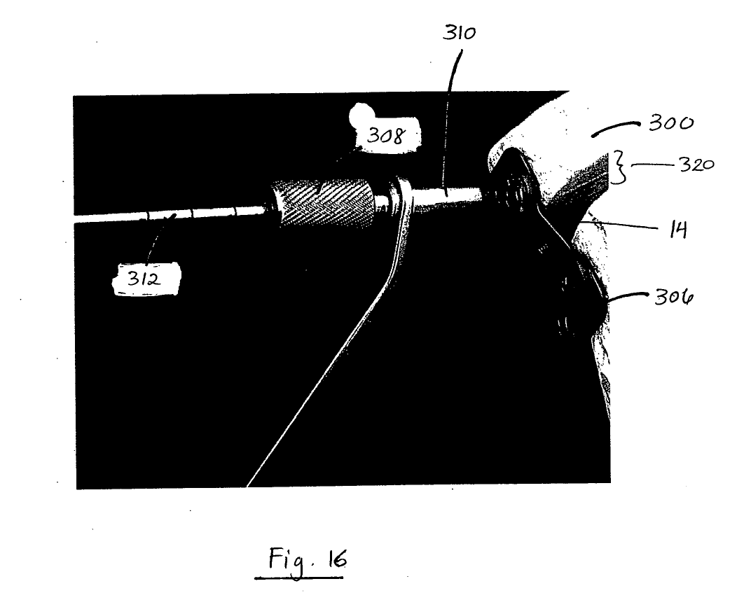

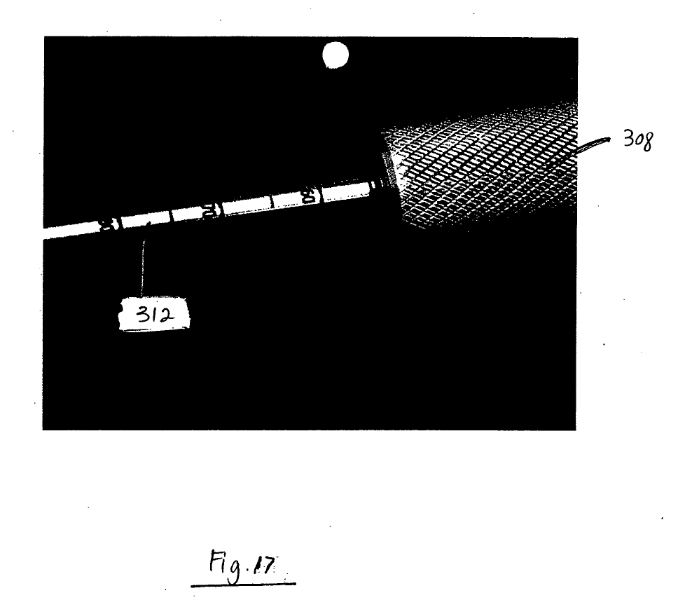

Referring to FIGS. 13-16, a hex-drive cortical plate screw 200 features a screw body 202 having a threaded bone portion 204 and a threaded screw head 206. The screw body 202 supports a cortical bone thread 208 that extends over the threaded bone portion 204, and a plate thread 210 that extends over the threaded screw head 206. The screw body 202 and cortical bone thread 208 have substantially uniform outer diameters over the threaded bone portion 204. In the transition between the threaded bone portion 204 and threaded screw head 206, the outer diameters of screw body 202 and plate thread 210 taper outwardly toward the head end of the plate screw 200 to facilitate initial engagement with the internal threads of washers 50 (FIGS. 1-3), followed by spreading of washers 50, as described further below The threaded bone portion 204 features about fifteen thread flights, preferably at a thread pitch of about 0.069, suitable for use in cortical bone. The threaded screw head 206 features about two thicker thread fights, preferably having a pitch of about 0.133, to complement the internal threads on washers 50. Referring to FIG. 15, preferably, a notch 212 is formed near the tip of screw 200 to facilitate self-tapping installation of screw 200 into bone. Referring to FIG. 16, a visual mark, such as black mark 214, is provided on the head of each screw 200. Bone screw 200 is made of metal, preferably titanium alloy.

An exemplary method of using the opening wedge osteotomy plate 10, 70, 80 and the bone plate screws 100, 200 is described below with reference to FIGS. 17-25, which illustrate a schematic view of a tibia in which an open wedge osteotomy is performed according to the present invention. In the following embodiment, an opening wedge tibial osteotomy is performed (shown completed in FIG. 25) with a selected bone plate 306 in a resecting tibia 302.

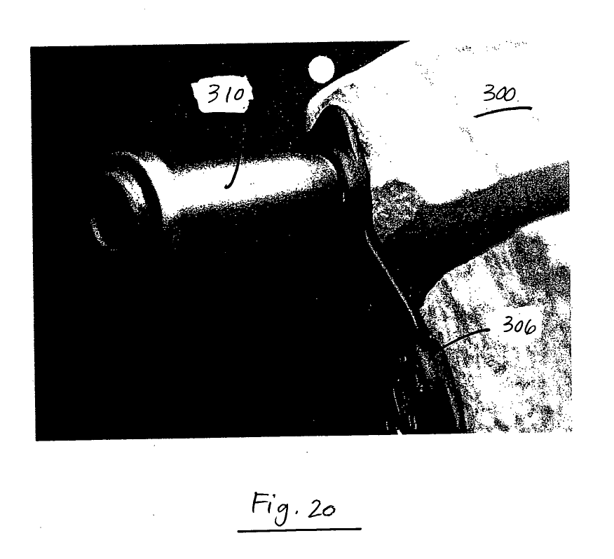

Referring to FIG. 17, the opening wedge tibial osteotomy begins by resecting tibia 300 so as to leave a bony hinge 302. A plate trial bar 304 is utilized to estimate the size of the bone plate that is needed for the desired correction 320. FIG. 19, for example, shows a 7.5 mm correction.

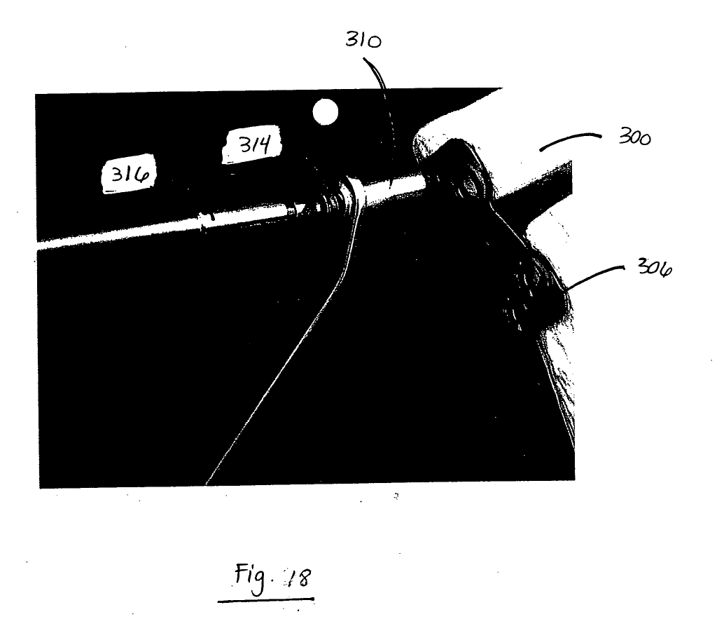

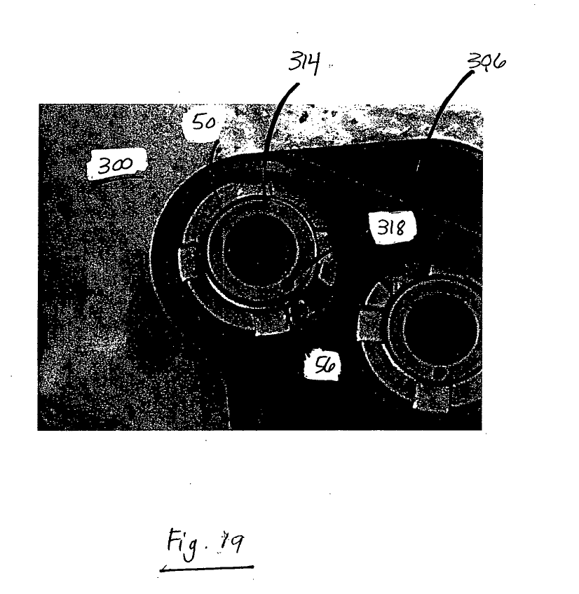

Referring to FIG. 18, the selected bone plate 306 (such as tibial osteotomy plate 10) having a projecting tooth 14 size equal to that measured by the plate trial bar 304 is positioned into the bone correction 320, ensuring optimum bone-to-plate contact with tooth 14. Prior to insertion, all of the washers 50 inside the plate are inspected and the washer teeth 52 are visualized to be on the side of the plate opposite the tooth 14. A drill guide 308 is inserted into a locking guide 310, and then into the opening in a washer 50 in plate 306. The locking guide 310 features complementary teeth which engage slots 52 on washers 50.

Referring to FIG. 19, a drill 312 is inserted under x-ray in standard fashion and drilled to (but not through) the lateral tibial cortex. The length of a screw may be determined by viewing laser lines formed on the back end of the drill 140. In FIG. 19, for example, a screw having a length of 55 mm is indicated.

Referring to FIG. 20, the drill guide 308 is removed while the position of the locking guide 310 is maintained in the slots 52 of washer 50. A plate screw 314 (such as cancerous plate screw 100 or cortical plate screw 200) with the appropriate diameter and length (for example, as per FIG. 19, a length of 55 mm is indicated) is installed through the locking guide 310 and into the bone using a driver 316.

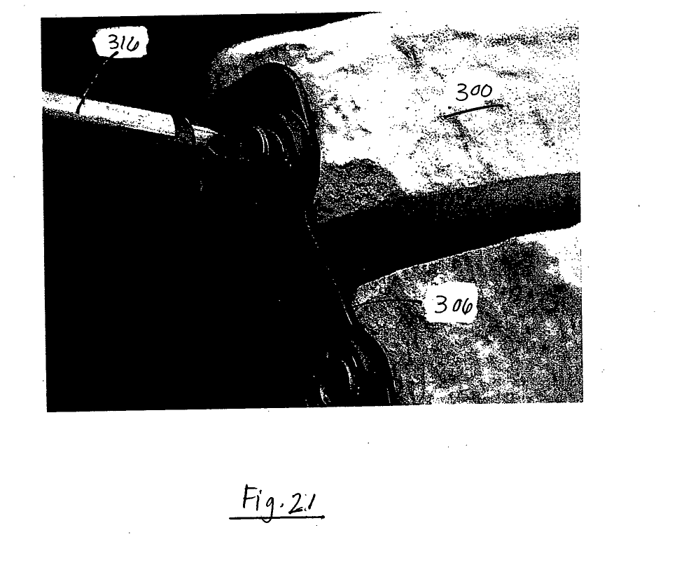

Referring to FIG. 21, just as the screw head threads contact the washer 50, the mark 56 on the washer 50 is positioned just clockwise to the black mark 318 on the screw head. Advantageously, the washer 50 can be rotated, if necessary, using the locking guide 310. Positioning the black marks in this way will ensure that the threads of the screw thread properly into the washer. The screw is advanced until it is tight and flush with the washer. As the screw 314 is advanced, the outward taper of the screw head spreads the split-sleeve washer 50, causing the assembly of the screw 314 and the washer 50 to become locked from rotation due to the frictional interference between an outer surface of the washer 50 and an inner surface of the bone plate 304 where the washer is inserted. The process is repeated for each of the screws until the bone plate is secured to the bone.

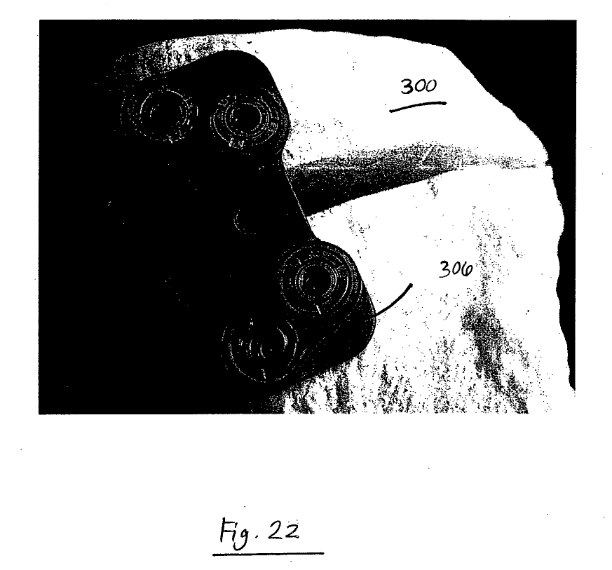

Referring to FIGS. 22 and 23, if the plate 306 is slightly proud from the cortical surface of the bone, the screws 314 can be unscrewed to slightly loosen the screw. In FIG. 22, the locking guide 310 is inserted into the slots 52 on the washer 50, and the washer is rotated clockwise to seat the plate 306 against the bone. FIG. 23 illustrates that the screw 314 is then advanced with the driver 316 until it is tight and flush with the washer 50. A completed installation of tibial bone plate 306 is illustrated in FIG. 24.

Although the present invention has been described in connection with preferred embodiments, many modifications and variations will become apparent to those skilled in the art.

1. A bone plate for performing opening wedge osteotomies in which a bone is resected so as to have an opening on one side governed by a bony hinge on the other side, the bone plate comprising:
a plate having opposing ends and internal wails defining holes through the plate;
a projection extending from the plate for insertion into the opening to prevent closure of the opening, the projection having opposed edges for engaging the bone to prop open the bone opening, the plate extending toward the ends on either side of the projection for engaging an outer surface of the bone; and
internally-threaded, split-sleeve washers disposed rotatably within the internal walls of the plate.

2. The bone plate of claim 1, wherein the split-sleeve washers include teeth projections defining slots formed on an outer side of each washer for engaging a washer guide.

3. The bone plate of claim 2, further comprising an alignment mark on the outer side of each washer.

4. A system for performing opening wedge osteotomies in which a bone is resected so as to have an opening on one side governed by a bony hinge on the other side, the system comprising:
a bone plate including:
a plate having opposing ends and internal walls defining holes through the plate;
a projection extending from the plate for insertion into the opening to prevent closure of the opening, the projection having opposed edges for engaging the bone to prop open the bone opening, the plate extending toward the ends on either side of the projection for engaging an outer surface of the bone; and
internally-threaded, split-sleeve washers disposed rotatably within the internal walls of the plate; and
plate screws for installation through the split-sleeve washers and into bone, the plate screws having a threaded bone screw portion for engaging bone, and a threaded screw head for engaging the internal threads of the split sleeve washers.

5. The system of claim 4, wherein the thread pitch of the threaded bone screw portion is different from the thread pitch of the threaded screw head.

6. The system of claim 5, further comprising alignment marks formed on an outer surface of each washer and on the head of each screw.

7. A method of performing an osteotomy comprising:
resecting a bone so as to have an opening on one side governed by a bony hinge on the other side;
installing a bone plate to maintain the bone opening bone, the bone plate including:
a plate having opposing ends and internal walls defining holes through the plate;
a projection extending from the plate for insertion into the opening to prevent closure of the opening, the projection having opposed edges for engaging the bone to prop open the bone opening, the plate extending toward the ends on either side of the projection for engaging an outer surface of the bone; and
internally-threaded, split-sleeve washers disposed rotatably within the internal walls of the plate; and
installing plate screws through the split-sleeve washers and into bone to secure the bone plate to the bone, the plate screws having a threaded bone screw portion for engaging bone, and a threaded screw head for engaging the internal threads of the split sleeve washers, the plate screws spreading the split-sleeve washers to provide frictional interference between the washers and the bone plate.
